(19) United States
(12) Patent Application Publication
(10) Pub. No.: US 2002/0068103 A1
(43) Pub. Date: Jun. 6, 2002

(54) Constitution improving agent for horses and method of improving constitution of horses

(75) Inventors: Nobushige Ishida, Tochigi (JP); Makoto Saito, Chiba (JP); Norikazu Fujii, Chiba (JP); Shoichi Tokutake, Chiba (JP)

Correspondence Address: YOUNG & THOMPSON, 745 SOUTH 23RD STREET 2ND FLOOR, ARLINGTON, VA, 22202

(73) Assignee: Kikkoman Corporation

(21) Appl. No.: 09/972,946

(22) Filed: Oct. 10, 2001

(30) Foreign Application Priority Data
Oct. 12, 2000 (JP) 2000-311969

(51) Int. Cl. A61K 35/78; A61K 38/00

(52) U.S. Cl. 424/766; 514/21; 514/801

(57) ABSTRACT

A constitution improving agent for horses comprising collagen, preferably in combination with a proanthocyanidin.

### BACKGROUND OF THE INVENTION

1. Field of the Invention

The present invention relates to a constitution improving agent for horses comprising collagen, a constitution improving agent for horses comprising collagen and a proanthocyanidin, and a method of improving the constitution of a horse comprising administering these constitution improving agents.

2. Description of the Related Art

Indices for evaluating the health condition of horses, such as riding horses and race horses, include gloss of a coat (hair), the growth condition and gloss of hooves, etc. Various constitution improving agents and nutrients for horses have been used for the purpose of improving these health conditions, strengthening muscles, increasing blood cells, accelerating healing of an injury, preventing infection in case of an injury, or deterioration of an injury, and the like. Known constitution improving agents or nutrients include vegetable proteins such as soybean meal, cotton seed meal, and peanuts meal; vitamins; minerals; and herbs such as garlic.

In recent years, intake of collagen (gelatin), which is an animal protein, has been attracting attention for its effects on health and beauty, more specifically, in preventing hypertension, alleviating osteoporosis, curing arthritis, rejuvenating the skin, and improving moisture retention of the skin. However, herbivorous horses have not been given animal proteins such as collagen (gelatin).

### SUMMARY OF THE INVENTION

An object of the present invention is to provide a novel constitution improving agent for horses, including riding horses and race horses, and a method of improving the constitution of horses which improve the gloss of hair and the growth condition or gloss of hooves or maintain or improve the physical conditions.

As a result of extensive investigations, the present inventors have found collagen (gelatin) or a combination of collagen and a proanthocyanidin available for achieving the above object.

The present invention relates to a constitution improving agent for horses which comprises collagen or a combination of collagen and a proanthocyanidin. It also relates to a method for improving the constitution of horses comprising administering the agent.

### DETAILED DESCRIPTION OF THE INVENTION

Collagen which can be used in the present invention is a fibrous protein which forms the largest proportion, i.e., 20 to 40%, in the total protein content making up an animal body. It is the main component constituting the connective tissue such as skin, bones, cartilage and tendons. Of the total collagen content, about 40% is present in the skin, about 20% in bones and cartilage, and the balance distributed throughout the body, e.g., in blood vessels and internal organs.

Collagen has a triple helix structure. Its amino acid composition comprises glycine in a proportion of one-third so that glycine appears once per three amino acids in the amino acid sequence. Being the simplest amino acid, glycine is little restrictive on molecular chain arrangement and greatly contributory to regeneration of the helix structure on gelling. Imino acids (e.g., proline and hydroxyproline) also constitute a large proportion, two-ninth in the total amino acid content. Having a pyrrolidine ring, imino acids are highly restrictive on the molecular chain arrangement and contributory to stabilization of the helix structure contrary to glycine.

On being heated, collagen undergoes changes in its triple helix structure and is separated into three random water-soluble molecules. The collagen thus thermally denatured and water-solubilized is gelatin. Collagen (gelatin) is produced on a worldwide industrial scale mainly from cowhide, pigskin and cow bones. Cowhide and pigskin allegedly form a proportion of about 70%, and cow bones about 30%, in the total raw material.

Collagen (gelatin) is commercially available under trade names of Atelo Helogen (available from Nippon Rika Co., Ltd.), Water-soluble Gelatin U (available from Nitta Gelatin Inc.), Water-soluble Collagen Peptide PA (available from Kyowa Hakko Kogyo Co., Ltd.), Gelita-sol D/1 (available from Newtrinova Japan). These commercial collagen products can be made use of in the present invention. The origin of collagen to be used in the invention may be either mammals or marine animals such as fishes.

Proanthocyanidins used in the present invention are condensed tannin present in various plant parts, i.e., a group of condensation or polymerization compounds comprising, as a constituent unit, flavan-3-ol or flavane-3,4-diol. These compounds release anthocyanidins, such as cyanidin, delphinidin, and pelargonidin, upon being treated with an acid, on which the above naming is based. Proanthocyanidins include procyanidin, prodelphinidin, propelargonidin, and the like which may be oligomers made up of two, three or four units or polymers made up of ten or more units; and stereoisomers thereof. They are obtained by extracting various plant parts, such as grape seeds, cranberry fruit, apple fruit, adztuki beans (red beans), and bark of sugi trees (Japanese cedar), hinoki trees (Japanese cypress) or pine trees.

These proanthocyanidins are commercially available under trade names of Gravinol (available from Kikkoman Corp.; prepared from grape seeds), Applephenon (available from The Nikka Whisky Distilling Co., Ltd.; prepared from immature apple fruit), and Pycnogenol (available from Horphag Research Ltd., GB; prepared from bark of maritime pine trees).

The dose forms of the constitution improving agent according to the present invention are not limited and include powdered forms, liquid forms and solid forms. For example, collagen (gelatin) can be sprinkled as such on feed, e.g., fodder. It may be dry blended with other nutrient components or be served as dissolved in a solvent such as water or in a solid form such as tablets. Where a proanthocyanidin is used in combination, it can be dry blended with collagen (gelatin) beforehand, and the mixture may be sprinkled directly on feed, e.g., fodder. Or a proanthocyanidin can be formulated into liquid or solid together with collagen (gelatin) in the same manner as described above.

The concentration of collagen (gelatin) in the constitution improving agent is preferably 5 to 100% by weight, still preferably 20 to 80% by weight. Where a proanthocyanidin is used in combination, its concentration in the constitution improving agent is preferably 5 to 95% by weight, still preferably 20 to 80% by weight.

A daily dose of the constitution improving agent is usually 0.05 to 5 g, preferably 0.1 to 3 g, in terms of the total amount of collagen (gelatin) and a proanthocyanidin per 50 kg-body weight.

The constitution improving agent according to the present invention has the following effects.

(1) Collagen (gelatin) improves the gloss of hair and hooves of a horse. Addition of a proanthocyanidin boosts the improvement.

(2) Collagen (gelatin) enhances superoxide dismutase (hereinafter abbreviated as SOD)-like activity in blood. Addition of a proanthocyanidin further increases the SOD-like activity. This effect results in suppression of an increase of serum peroxylipid, which is believed to suppress damage of muscular cells by active oxygen and to enhance recovery of muscles from fatigue.

The effects of the present invention will be demonstrated in the following Test Examples.

### TEST EXAMPLE 1

### Visual Observation on Gloss of Hair and Condition and Gloss of Hooves

Sixteen horses (Thoroughbreds; owned by Japan Racing Association) each weighing 450 to 520 Kg were divided into four groups A to D each consisting of four horses and given feed (oats) to which an additive was added as described below once a day for consecutive one month.

Group A: feed with no additive

Group B: feed containing 6 g/horse of collagen (Water-soluble Collagen U, available from Nitta Gelatin Inc.)

Group C: feed containing 6 g/horse of collagen (Water-soluble Collagen U) +6 g/horse of proanthocyanidin (Gravinol, available from Kikkoman Corp., grape seed extract; proanthocyanidin content: 40% by weight or more)

Group D: feed containing 6 g/horse of proanthocyanidin (Gravinol)

After two weeks and after one month from the start of dieting, the gloss of the hair and the hooves and the condition of the hooves were observed with the naked eye. The results obtained are shown in Table 1 below.

TABLE 1

| Group | After 2 Weeks | After 1 Month |
|---|---|---|
| A | − | − to + |
| B | − to + | + to + + |
| C | + to + + | + + to + + + |
| D | The diet was refused. | The diet was refused. |

Note:
−: No change
+: Slight improving effects were observed.
+ +: Improving effects were observed.
+ + +: Significant improving effects were observed.

The results in Table 1 prove that group B which was given 6 g of collagen shows improvement in gloss of hair and hooves over group A which was given no additive and that group C which was given 6 g of collagen and 6 g of a proanthocyanidin shows further improvement over group B. Group D refused to take the feed containing 6 g of a proanthocyanidin so that the evaluation on the hair and the hooves was not made.

The feed containing the proanthocyanidin was refused by horses on account of astringency of the proanthocyanidin, but the feed containing the proanthocyanidin in combination with collagen was accepted probably because the astringency of the proanthocyanidin could be masked by collagen.

### TEST EXAMPLE 2

### Determination of SOD-like Activity

Blood was taken from the jugular vein of group B (placed on collagen) and group C (placed on collagen and a proanthocyanidin) before the administration and after one month from the start of the administration. The SOD-like activity of the serum was determined with an ESR measuring apparatus. The results obtained are shown in Table 2 below.

TABLE 2

| | SOD-like Activity Intensity | SOD-like Activity Intensity after |
|---|---|---|
| | before Administration | 1 Month |
| | (cell mean) | (cell mean) |
| Group B | 0.998 ± 0.186 | 2.551 ± 0.935 |
| Group C | 1.173 ± 0.345 | 3.964 ± 1.153 |

It is seen that the serum SOD-like activity increased in group B and that the increase in SOD-like activity achieved in group C is almost twice that in group B.

### TEST EXAMPLE 3

### Effect on Peroxylipid Level in Serum after High Intensity Exercise

Four horses (Thoroughbreds; owned by Japan Racing Association) each weighing 450 to 520 Kg which were put into usual training for running were made to take a high intensity exercise using a treadmill. Blood was drawn before and after the exercise to determine peroxylipid level in the serum. From this day, the horses were maintained on the same diet containing 6 g/day of collagen (Water-Soluble Gelatin U) and 6 g/day of a proanthocyanidin (Gravinol) as used in Test Example 1. After 3 weeks' feeding, the horses were made to take the same high intensity exercise on the treadmill, and the blood was drawn before and after the exercise to determine the serum peroxylipid level. The percent increases in serum peroxylipid level due to the exercise before and after the maintained administration of the additives are shown in Table 3 below. Incidentally the four horses showed no significant differences in lactic acid level in the serum after the exercise which is indicative of the exercise load.

TABLE 3

| | Percent Increase in Serum Peroxylipid (%) | |
|---|---|---|
| | Before Administration | After Administration |
| | of Additives | of Additives |
| Collagen 6 g + | 69.5 ± 63.8 | 9.0 ± 23.75 |
| Proanthocyanidin 6 g | | |

A maintained administration of collagen combined with a proanthocyanidin was proved to suppress an increase in serum peroxylipid level in race horses. As demonstrated in Test Examples 2 and 3, the constitution improving agent comprising collagen (gelatin) enhances SOD-like activity in horse blood, and the constitution improving agent comprising collagen (gelatin) and a proanthocyanidin exhibits more potent SOD-like activity. Accordingly, the constitution improving agent of the present invention is believed to suppress muscular cell damage in horses due to active oxygen and to help muscles recover from fatigue.

### TEST EXAMPLE 4

To seven horses Thoroughbreds) each weighing 450 to 520 kg, a mixture of an equal amount of collagen powder (Water-Soluble Gelatin U, available from Nitta Gelatin Inc.) and proanthocyanidin powder (Gravinol, available from Kikkoman Corp.) was dosed as a constitution improving agent in an amount of 12 g/day per horse for consecutive one month by sprinkling the mixture to the fodder. During the dose of the mixture, the horses were made to take the following exercise every morning.

| | |
|---|---|
| Warming-up | walk for 30 minutes |
| Entering into the racetrack | trot for 1000 m |
| | canter for 3000 m |
| | gallop for 1000 m |

Within ten minutes after the exercise finishes, blood was drawn to determine the cleatine phospho kinase (CPK) activity. The following Table 4 shows the results.

TABLE 4

| | CPK activity (IU/L, | CPK activity (IU/L, |
|---|---|---|
| | 37 ® C) before | 37 ® C) after one |
| | Administration | month |
| Horse 1 | 291 | 238 |
| Horse 2 | 282 | 228 |
| Horse 3 | 334 | 254 |
| Horse 4 | 276 | 242 |
| Horse 5 | 284 | 269 |
| Horse 6 | 250 | 229 |
| Horse 7 | 296 | 250 |
| Average | 287 | 244 |

As seen from the Table 4, the increase of CPK activity was suppressed in every horse, which demonstrates that a mixture of collagen and proanthocyanidin exhibits advantageous effect on improving the constitution of horses.

CPK: an enzyme which plays an important role for energy metabolism of muscle cells such as skeletal muscles and cardiac muscles. Contained in, other than these muscles, smooth muscles and brain, but seldom exists in blood cells or a liver. However, once the tissue cells of the muscles are damaged caused by the exercise, CPK activity in the blood increases. Thus, the determination of serum CPK activity is useful as an indication of whether or not the tissue cells such as muscle and brain are damaged.

### FORMULATION EXAMPLE 1

Liquid Constitution Improving Agent for Race Horses

| | |
|---|---|
| Collagen powder (Water-Soluble Gelatin U, | 20% |
| available from Nitta Gelatin Inc.) | |
| (by weight, hereinafter the same) | |
| Proanthocyanidin powder | 20% |
| (Gravinol, available from Kikkoman Inc.) | |
| Sugar | 5% |
| Glucose | 8% |
| Minerals | 5% |
| Guar gum | 2% |
| Purified water | 40% |

The above ingredients were mixed up by stirring to prepare a liquid constitution improving agent for race horses. The liquid preparation can be given to horses by sprinkling on feed such as fodder in an amount of 15 to 35 ml.

### FORMULATION EXAMPLE 2

Powdered Constitution Improving Agent for Race Horses

| | |
|---|---|
| Collagen (Water-Soluble Gelatin U, | 40.5% |
| available from Nitta Gelatin Inc.) | |
| Proanthocyanidin powder (Gravinol, | 40.5% |
| available from Kikkoman Inc.) | |
| Sugar | 5% |
| Glucose | 8% |
| Minerals | 4% |
| Guar gum | 2% |

The above ingredients were mixed up by stirring to prepare a powdered constitution improving agent for race horses. The powder can be given to horses by sprinkling on feed such as fodder in an amount of 7 to 17 g.

### FORMULATION EXAMPLE 3

Constitution Improving Agent (Tablets) for Race Horses

| | |
|---|---|
| Collagen (Water-Soluble Gelatin U, | 20% |
| available from Nitta Gelatin Inc.) | |
| Proanthocyanidin powder (Gravinol, | 20% |
| available from Kikkoman Inc.) | |
| Sugar | 5% |
| Glucose | 8% |
| Minerals | 4% |
| Starch | 38% |
| Casein | 5% |

The above ingredients were mixed up by stirring and punched into tablets for oral administration.

What is claimed is:

1. A constitution improving agent for horses which comprises collagen.

2. The constitution improving agent for horses according to claim 1, wherein collagen is gelatin.

3. The constitution improving agent for horses according to claim 1, wherein collagen is present in an amount of 5 to 100% by weight.

4. The constitution improving agent for horses according to claim 1, which further comprises a proanthocyanidin.

5. The constitution improving agent for horses according to claim 4, wherein said proanthocyanidin is an extract of grape seeds.

6. The constitution improving agent for horses according to claim 4, wherein said proanthocyanidin is present in an amount of 5 to 95% by weight.

7. A method of improving the constitution of a horse which comprises administering collagen.

8. The method according to claim 7, wherein collagen is administered at a dose of 0.05 to 5 g per 50 kg of the body weight of a horse per day.

9. A method of improving the constitution of a horse which comprises administering collagen and a proanthocyanidin.

10. The method according to claim 9, wherein collagen and the proanthocyanidin are administered each at a dose of 0.05 to 5 g per 50 kg of the body weight of a horse per day.
